(19) United States
(12) Patent Application Publication
(10) Pub. No.: US 2017/0208375 A1
(43) Pub. Date: Jul. 20, 2017

(54) APPARATUS, METHOD AND SYSTEM FOR DISTRIBUTED CHEMICAL OR BIOLOGICAL TO DIGITAL CONVERSION TO DIGITAL INFORMATION USING RADIO FREQUENCIES

(71) Applicants: John William Hodges, JR., Ocoee, FL (US); Marc Edward Rippen, Treasure Island, FL (US)

(72) Inventors: John William Hodges, JR., Ocoee, FL (US); Marc Edward Rippen, Treasure Island, FL (US)

(21) Appl. No.: 15/422,462

(22) Filed: Feb. 1, 2017

Related U.S. Application Data
(63) Continuation-in-part of application No. 14/884,092, filed on Oct. 15, 2015.
(60) Provisional application No. 62/082,735, filed on Nov. 21, 2014.

(51) Int. Cl. H04Q 9/00; G01N 27/416; G01N 33/02; G01N 27/12

(52) U.S. Cl. CPC H04Q 9/00; G01N 27/12; G01N 27/416; G01N 33/02; H04Q 2209/47; H04Q 2209/886; H04Q 2209/845

(57) ABSTRACT

A system including at least one resonator located within a first electrical dielectric medium where a product is located, the product emits at least one chemical as the product ages, and transmitter located at a dielectric boundary of said first electrical dielectric medium to provide communications to an external analyzer of data emitted by the at least one resonator and to provide electrical energy to the resonator by way of radio waves.

### CROSS-REFERENCE TO RELATED APPLICATIONS

This is a Continuation-In-Part of U.S. application Ser. No. 14/884,092 filed Oct. 15, 2015, which claims benefit of U.S. Provisional Application No. 62/082,735 filed Nov. 21, 2014, and incorporated herein by reference in their entirety.

### BACKGROUND OF THE INVENTION

Embodiments relate to temperature, chemical and biological sensing RFID tags. Every day, millions of tons of perishable goods are produced, transported, stored or distributed worldwide. These products are considered perishable because the internal biological and chemical processes continue within perishable goods after harvesting or manufacture. These products can be food items, such as, but not limited to, fruit, vegetables, flowers, fish, meat and dairy products or medical products like drugs, blood, vaccines, organs, plasma and tissues. Some chemicals and electronic components are also sensitive. All of these products can have their properties or quality change rapidly when faced with inadequate environmental conditions during transport or storage. Even with today's network of refrigerated rail cars, trucks, and intermodal containers, shippers of fresh produce, frozen foods, temperature-sensitive liquids, and other perishable goods face numerous hurdles to ensure that their products arrive on time, unspoiled, and with adequate remaining shelf life. In the industry, this network is called the Cold Chain.

For just one product sector, reports has concluded that the cost of food spoilage totals more than $35 billion annually. Produce is a living, breathing commodity, which emits heat and carbon dioxide. The risks of a failure at any point in the process from field to table can cause excessive ripening, weight loss, softening, color and texture changes, physical degradation and bruising, and attack by rot and molds. These factors affect freshness, desirability, and marketability. Strict temperature control throughout the supply chain can minimize the risk of food-borne illnesses because low temperatures drastically reduce the growth rate of most human pathogens.

In the United States (“U.S.”) alone, spoilage and waste in the food supply chain result in the loss of 23% to 25% of fruits and vegetables post-harvest, according to ChainLink Research. Furthermore, the research firm reports that 25% to 50% of the total economic value is lost because of reduced quality of products in the supply chain. There are less tangible costs as well such as: the costs of being out-of-stock of an item due to unplanned spoilage; the cost of overstocking items to avoid being out-of-stock; lost margin from price discounting and brand damage from lack of trust in product shelf life.

When these products fail to make the grade, they turn into waste. For years, having products simply turn to waste has been considered a cost of doing business. For business, these perishable goods invariably contribute the highest income; ironically, they are also responsible for the highest level of waste and economic loss. Waste does not scale well, the larger the organization, the greater percentage of products lost to waste than at a smaller organization.

The causes of the spoilage and waste losses are numerous, but about half the loss is due to variations in temperature and half to other processes. Waste begins in the field or factory and needs to be managed from harvest or manufacturing through to delivery to the retail store. Every year in the U.S., there are approximately 7.59 million truckloads of perishable goods carried on 182 million pallets in 4.37 billion boxes with values up to $85,000 per pallet. This is a very large logistics problem spanning the U.S., Canada and Mexico and margins are razor thin in the shipping industry.

To truly measure a products condition though the supply cold chain direct biological and chemical measurements of the “fingerprints” of decay and contamination are required. For economic reasons direct biological and chemical measurements of these “fingerprints” are simply not possible due to sensor costs. While the most accurate, conventional laboratory analysis using manual lot sampling is skilled labor intensive, has very high equipment costs, the slowest time to answer and is simply too expensive.

### STATE OF THE ART

RFID technology in comparison to barcodes in terms of memory capacity, readability, speed, being re-programmable, robustness and scalability is vastly superior, but conventional RFID does not penetrate water based fruits, vegetables or meats very well because these materials are a lossy dielectric and present an abrupt dielectric shift to impinging radio waves. FIG. 2 is presentative of the issues that are encountered with RFID technology.

Currently the aim of all existing prior art approaches is focused on implementing the electronic product code (“EPC”) Global Tag Data Standard (TDS) into sensor RFID tags, which have the same capabilities as the conventional RFID technology is already providing for other product types.

However, at the carton and at items levels, conventional RFID and RFID concepts cannot work for produce. The reason for this is that conventional RFID is about single pass read speed and accuracy whereas the chemical measurements take too much time. The delay in time is due to chemical ionic transfer processes not electronic transfer processes so the time scales are six orders of magnitude slower.

Carton and item level tagging is desired because it allows much higher granularity of the Chemical and Biological measurements (e.g. it allows measurement at the individual package level rather than the pallet level in current devices. In recognition of this problem in the “Cold Chain”, current RFID solutions providers are driving the current market push. However, current RFID Technology has reached a technological plateau. This is not for lack of trying. This is demonstrated by the introduction of semi-active or battery operated passive (“BOP”) temperature logging RFID tags as a stopgap.

Other entities realizing this problem have recently introduced semi-active or BOP temperature logging RFID in an attempt to increase granularity. However, because of their cost, the data granularity of these RFID temperature-logging sensors is poor because they can only be fitted to selected areas. Care must be taken in placing these sensors in enough numbers and in locations that are representative of the entire product load. The thermal behavior of transport and storage systems varies significantly depending on the type of product, stowage practices, packaging and many other factors leading to a wide spatial variation of product cargo temperatures. The spatial variation problem has not been solved notwithstanding a great deal of time and money.

A feature of the inventive concept is that it allows much higher granularity of the Chemical and Biological measurements (e.g. it allows measurement at the individual Package level rather than the Pallet or Truck Load level in current devices.

A further problem with both the Active and “Semi-passive” temperature sensing RFID tags is they can only infer the amount biological and chemical decay based on historical data logs. It is obvious direct biological and chemical measurement of the decay process is preferable to any estimate based on temperature history.

Temperature logging is inferior in every respect because it can only estimate based on historical data. Historical data for each product is simply not available so existing devices are currently expending R&D capital to build the required data sets.

Most importantly, while the causes of the spoilage and waste losses are numerous only about half the losses are to variations in temperature. A solution is required to close this gap by providing information about the other half.

### BRIEF DESCRIPTION OF THE INVENTION

The present invention provides a set of two apparatus for sensing the concentration of a chemical or biological analyte and the chemical or biological analyte temperature, for communicating data derived thereof and for acting as radio frequency identification (RFID) tag, wherein the sensor RFID tags are in direct contact with the analyte to be measured. A system is disclosed that comprises at least one resonator located within a first electrical dielectric medium where a product is located, the product emits at least one chemical as the product ages, and transmitter located at a dielectric boundary of said first electrical dielectric medium to provide communications to an external analyzer of data emitted by the at least one resonator and to provide electrical energy to the resonator by way of radio waves.

### BRIEF DESCRIPTION OF THE DRAWINGS

A more particular description briefly stated above will be rendered by reference to specific embodiments thereof that are illustrated in the appended drawings. Understanding that these drawings depict only typical embodiments and are not therefore to be considered to be limiting of its scope, the embodiments will be described and explained with additional specificity and detail through the use of the accompanying drawings in which:

FIG. 1 Shows an illustrative diagram of the current state of the art;

FIG. 2 shows an illustrative diagram of an embodiment of a system;

FIG. 3 shows a block diagram of a resonator;

FIG. 4 shows an embodiment of the resonator;

FIG. 5 shows an embodiment of the resonator from another view;

6 shows a block diagram of Part B of the system;

FIG. 7 shows an embodiment of a system used with a pallet of product;

FIG. 8 shows an illustrative diagram of RF radiation patterns inside a pallet;

FIG. 9 shows a flowchart of an embodiment of a method;

FIG. 10 shows a flowchart of an embodiment of another method;

FIG. 11 shows a graphical illustration of the method in FIG. 10;

FIG. 12 shows a flowchart of an embodiment of a third method;

FIG. 13 shows a block diagram of a Hidden Markov Model;

FIG. 14 shows a graphical representation of temperature versus mission time; and

FIG. 15 shows a graphical representation of metabolite versus mission time.

### DETAILED DESCRIPTION OF THE INVENTION

Embodiments are described herein with reference to the attached figures wherein like reference numerals are used throughout the figures to designate similar or equivalent elements. The figures are not drawn to scale and they are provided merely to illustrate aspects disclosed herein. Several disclosed aspects are described below with reference to non-limiting example applications for illustration. It should be understood that numerous specific details, relationships, and methods are set forth to provide a full understanding of the embodiments disclosed herein. One having ordinary skill in the relevant art, however, will readily recognize that the disclosed embodiments can be practiced without one or more of the specific details or with other methods. In other instances, well-known structures or operations are not shown in detail to avoid obscuring aspects disclosed herein. The embodiments are not limited by the illustrated ordering of acts or events, as some acts may occur in different orders and/or concurrently with other acts or events. Furthermore, not all illustrated acts or events are required to implement a methodology in accordance with the embodiments.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

There is an opportunity for dramatically increased synergy between electronics and chemistry and biology, fostered by the march of electronics technologies and rapid advances in system, chemistry and biology to reduce waste during shipping.

The key enabling technology linking these two areas is a conversion technology that converts chemical or biological information into electrical signals and providing the ability to collect and analyze essential data on the state of the chemical analyte, biomolecules and cells (chemical, physical, structural, functional) wirelessly.

An object of an embodiment of the invention is to provide a solution to food spoilage and waste by providing stakeholders with actionable data about the quality and condition of their product as it moves through the supply chain in order to better manage waste from the field or factory to the customer automatically and wirelessly.

A further object of an embodiment of the invention is to eliminate electrochemical batteries in contact with a fresh food product as these devices can contaminate the food product.

These objects are achieved by providing an apparatus, method and system to enable direct biological, chemical and temperature measurements of the “fingerprints” of decay and contamination to truly measure a products condition though the supply chain. An inventive concept achieves a disposable sensor system at a fraction of their current costs combined with a new circuit and wireless communications techniques to achieve automation combined with accuracy and reliability to provide a chemical or biological sensor system at the carton and item level, which exhibits none of the problems with which prior sensors system are beset.

These objects are further achieved at an economic price point that allows disposability reducing cross contamination at the shipping carton and item level without expensive conventional laboratory analysis using manual lot sampling associated skilled labor, very high equipment costs and slow time to answer.

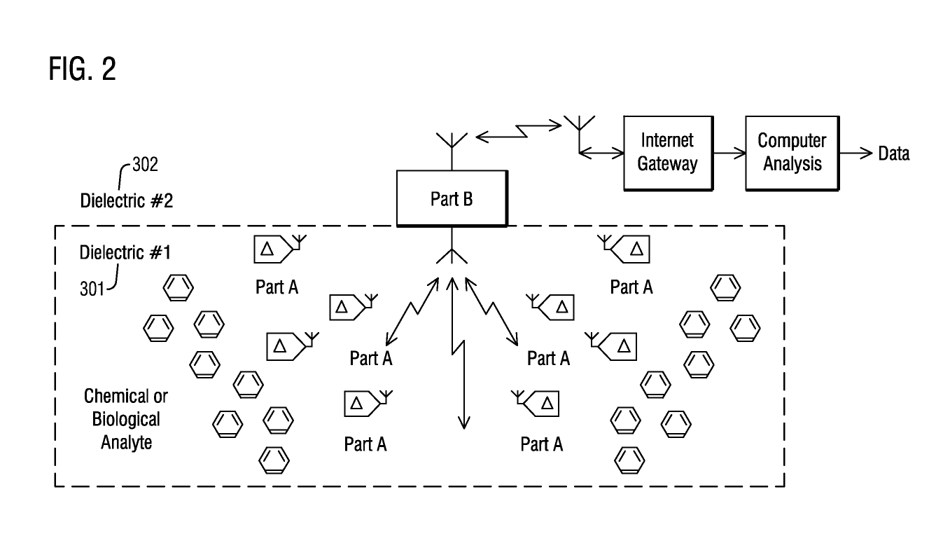

FIG. 2 shows an illustrative diagram of an embodiment of a system. The system 100 comprises a RFID tag system providing a set of two apparatus, a Part A and Part B for sensing a concentration of a chemical or biological analyte and the chemical or biological analyte temperature, communicating data derived thereof and a system acting as a radio frequency identification (RFID) tag, wherein the RFID tag is in direct contact with the analyte to be measured.

Part A may comprise a temperature sensor and a chemical sensor. Part A may also be considered a resonator. The sensors may be immersed in at least one of a liquid analyte, gaseous analyte or combination (chemical analyte) 115 thereof in an electrical dielectric medium of interest. Non-limiting examples of the dielectric medium may comprise the produce, such as, but limited to, produce, a gas, such as, but not limited to oxygen, surrounding the product, and another resonator within the produce area, as disclosed further herein. Part A may communicate, using radio waves, as a non-limiting example, to at least the Part B which may be located on a boundary of and/or perhaps, extending into the dielectric material at the boundary of the electrical dielectric mediums of interest.

Part B may comprise, in general, transmitter. As explained further herein, the transmitter at least comprises radio system. Also shown is a computing device to perform analysis of information obtain from Part A. As is further shown, two dielectric material are provided. A first dielectric material is within a location where measurements are being taken. A second dielectric material is outside of the location where measurements are being taken. Thus, Part A is within the first dielectric material whereas Part B is within the second dielectric material. As further shown a plurality of resonators, or Part A are expected to be used. They may collectively be referred to as a colony. A single Part B is used with respect to a particular colony.

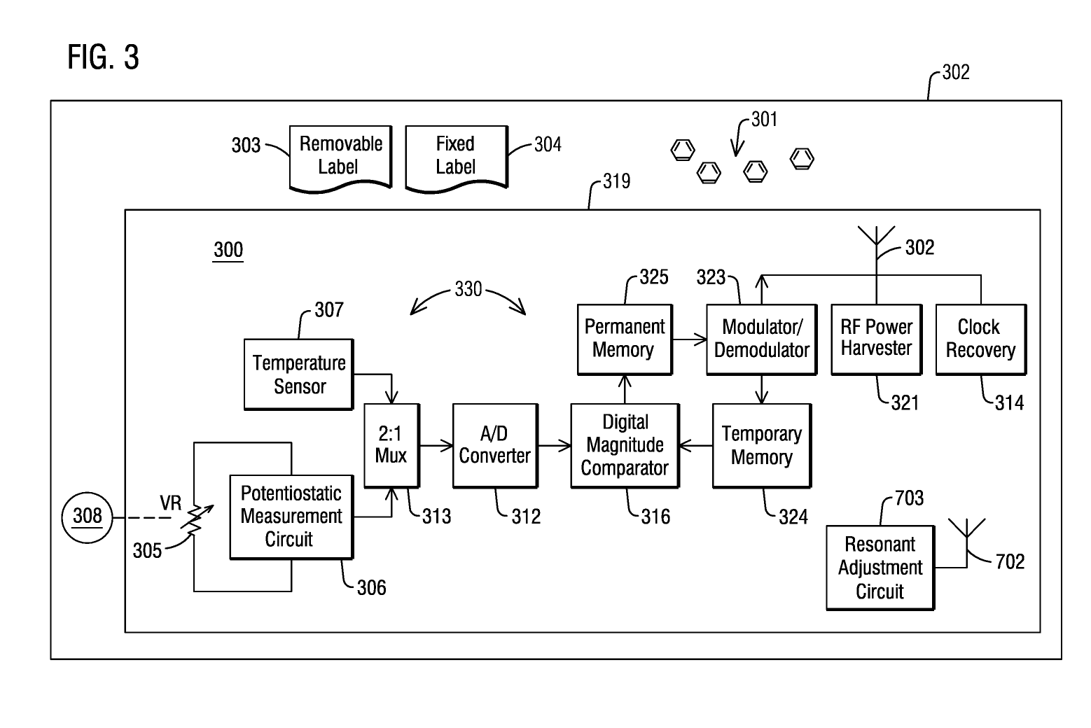

Referencing now FIG. 3, the Part A apparatus is disclosed. A liquid analyte or gaseous analyte 301 or combination (generally referred to as a chemical analyte 115) is shown which is in an electrical dielectric medium of interest. A removable label 303 and a fixed label 304 are also disclosed, which are attachable to an outer cover or surface of the Part A. A housing 301 provides a dielectric medium around the resonator 309. Though not shown, the housing also has at least one component to maintain alignment of the resonator under mechanical vibration 319, referred to as an anti-vibration support 319. More specifically, the resonator may be arranged where it has to maintain a certain position with respect to the produce. The components to maintain alignment are provided reduce vibrational effects, such as experienced during transport to misalign the resonator.

A mechanism 308 for delivering gaseous or a liquid analytes to the said area is provided. The analytes includes chemicals that are naturally emitted from the product as a normal part of its chemical processes. The chemical process is a non-reversible process as the process is due to time and age. This mechanism may be holes within outer cover. Within Part A, at least one antenna in the form of an electrically small magnetic dipole 302 is provided. Further shown is at least one first sensor comprising an area of a reversible chemoresistive surface VR 305 coupled to a potentiostatic measurement circuit 306. The potentiostatic measurement circuit 306 is coupled to a temperature sensor 307 by way a two-to-one multiplexer 313 which provides for switching when commanded.

An analog to digital converter 312 is provided. A clock recovery mechanism 314 is also provided. An electrical energy harvesting mechanism from rectified RF energy 321, acting as a modulator for RF modulation and as an envelope detector demodulator suitable for off-on-keying, (OOK) or amplitude shift keying, (ASK) modulation acting as a receiver for a reference value, commands and other signals as required 323, a circuit acting as a digital magnitude comparator 316, a circuit temporally holding digital data 324 were said data is a reference value and command and temporally holding digital data were said data is a measurement value; a circuit permanently holding digital data 325 were said data is an identification value said code value references said label and permanently holding digital data were said data is an encoding value; and a circuit temporally holding digital data were said data is a measurement value; and may, perhaps include additional variable capacitors 703 and least one second electrically small antenna in the form of a magnetic dipole inductively coupled to the first electrically small antenna 702.

The apparatus 300 may, possibly operate in a gaseous medium, or in a liquid medium isolated by a protective housing 309 creating an air space around the antenna 302. The apparatus 300 may, possibly be protected from mechanical crushing forces by the protective housing creating an air space around the antenna 302. The apparatus 300 may, possibly incorporate features that maintain optimum orientation from mechanical vibrational forces by a protective housing creating an air space around the antenna 302.

The circuit 330 may, possibly contain a silicon microchip and at least one sensor is configured to sense a specific analytes concentration or other such data deemed appropriate.

The antenna 302, which may be, but is not limited to, electrically small magnetic dipole antenna, may receive RF energy and to transmit the energy simultaneously wherein the signal is an electromagnetic radio frequency signal.

The first electrical dielectric medium of interest 301 has an electromagnetic wave number greater than or equal to three times said second dielectric medium of interest 302. Under this condition the step change in the dielectric causes the electromagnetic wave inside the first dielectric medium 301 to be reflected back into the first dielectric medium 301. It is important to note that this mechanism causes severe phase distortion of the electromagnetic signal and it is for this reason many of the features of the invention will be apparent to those art exist and are different than conventional radio practice.

Additionally, Part A, does not contain a battery or conventional oscillator circuit. Excluding these components provide for both lower costs and prevention of contamination of food products.

The sensor 330 is created using a chemoresistive surface 305 and senses a specific analytes concentration or other such data deemed appropriate.

Wireless operation is provided using a clock recovery circuit 314 which recovers clock information modulated on a RF signal, and also receives commands modulated on a RF signal from and only from apparatus Part B described below. On command this circuit acts on the combination of recovered clock information and commands to initiate and perform analog to digital conversion on a voltage signal generated from a potentiostatic measurement circuit 306 sensing a specific analytes concentration, temperature or other such data deemed appropriate. In another non-limiting example, the potentiostatic measurement circuit 306 may be replaced with a suitable galvanometric measurement circuit.

The digital output value from the A/D circuit 312, is digitally magnitude compared to a digital reference value temporarily stored in the temporary memory circuit 324, from the commands noted above and if the output of the digital magnitude comparator is logically TRUE the data from the circuit permanently holding digital data 325 is read out in a bitwise manner to said RF modulator structure or if logically FALSE no digital data is read out.

The above digital data stored in the memory 325 may provide for a unique identification code value, such as, but not limited to, 24 bits, encoded with code value that is a known orthogonal code or “gold” code at the time of manufacture. For security reasons, the size of the “gold” and the gearing ratio or modulo is variable providing an encryption mechanism where the encoding operation is accomplished using a logical Exclusive OR function though other functions can be used and is not reprogrammable in the field.

The output from the digital data stored in the memory 325 is used to drive a modulator thus creating RF modulation in an amplitude-shift keying or off-on-keying modulated signal representing the encoded digital representation of the identification code value where the identification code value references the printed label and to transmits the modulated signal using the antenna

The antenna 302 may acts as an electromagnetic RF summing device combining each tags unique RF modulation with a plurality of other similarly uniquely encoded tags RF modulation in supposition or summing as a response signal in electromagnetic space.

Note tags transmit only if the measurement value exceeds the commanded reference value, though obviously the apparatus can be designed to transmit only if the measurement value is less than the commanded reference value, logically the two operations are the same.

As briefly identified above, an identical operation is carried out for measuring temperature and switched using the two-to-one multiplexer 313 when commanded. All other functions remain the same.

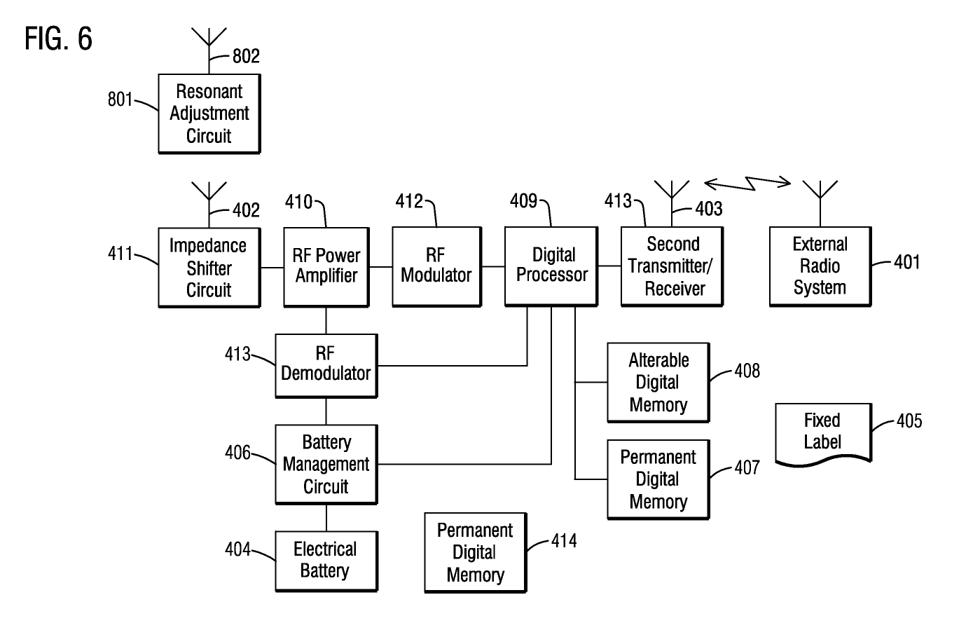

FIGS. 4 and 5 shows an embodiment of the resonator. The casing or cover 319 is shown. The sensor 330 and circuit is located within the cover 319. It is held in place by a first component to ensure orientation and non-movement within the cover 319. The components providing non-vibration support and orientation are also shown, though more clearly in FIG. 5. Also visible is the mechanism 308 for delivering, or allowing to pass, gaseous or a liquid analytes to the said area.

Referencing now FIG. 6, the Part B apparatus is shown. In general, a transmitter located at a dielectric boundary of the first electrical dielectric medium to provide communications to an external analyzer of data emitted by the at least one resonator and to provide electrical energy to the resonator by way of radio waves is shown. More specifically, Part B comprises an external radio system 401. A first antenna tuned to radio frequencies of Part A in the form of a feed suitable for exciting an electrically dielectric resonant structure 402 is shown. Also provided is a second antenna tuned to the external radio systems frequencies 403. An electrical battery 404, located outside of the housing of the product is shown. A fixed label 405 is also provided. A set of connections providing for the formation in the structure of layers of conducting material comprising a circuit is shown. A circuit 406 may be for power management from the battery 406. The overall circuit, or Part B, also may comprise a permanent digital memory 407. The overall circuit may comprising an alterable digital memory 408. The overall circuit may comprise a digital processor 409, Other components may include, but are not limited to a radio frequency power amplifier 410 connected to said first antenna tuned to radio frequencies of said apparatus first part connected to said feed suitable for exciting an electrically dielectric resonant structure, an impedance shifter circuit 411 to alter the impedance of the antenna tuned to radio frequencies of the Part A connected to the feed suitable for exciting an electrically dielectric resonant structure. a RF modulator 412 for off-on-keying, (OOK) or amplitude shift keying, (ASK) modulation acting as a transmitter for a reference value, command and clock. A RF Demodulator 413 may be provided to act as an envelope detector demodulator for off-on-keying, (OOK) or amplitude shift keying, (ASK) modulation acting as a receiver for encoded RF inductive coupling modulation in supposition or summing as a response signal from a plurality of said apparatus first part; A permanent digital memory 414 comprising a transmitter and receiver tuned to the external radio systems frequencies may be provided. An indicator light which may include an resonance adjustment circuit 801 and second electrically small antenna in the form of a magnetic dipole inductively coupled to the first electrically small antenna 802 may also be provided.

The Part B apparatus is located on the boundary of first electrical dielectric medium of interest 301 where this dielectric medium has an electromagnetic wave number greater than or equal to three times the second dielectric medium of interest 302. Additionally, there is no conductive surface at this boundary.

The Part B apparatus uses an antenna 402 as a feed. Mechanically this antenna slips in between individual boxes on a pallet in the preferred embodiment. Antenna 402 takes the form of an electrically small magnetic dipole tuned to radio frequencies of the apparatus part A where this feed excites an electrically dielectric resonant structure formed by the conditions noted above. This feed 402 is designed to be relocatable to accommodate differing pallet configurations with different dielectric properties.

To those skilled in the art it is apparent in cases where the dielectric medium 301 has an electromagnetic wave number less than three times the second dielectric medium of interest 302 a conductive surface at this boundary must be provided to ensure proper operation.

More specifically to Part B when considering it as a single circuit, the circuit structure comprises a radio frequency power amplifier connected to the first antenna 402 tuned to radio frequencies of the apparatus Part A exciting the electrically dielectric resonant structure. This circuit comprising a radio frequency power amplifier 410, the resonant structure described above and plurality of antennas 302 and RF power harvesters 321 provides electrical power to the plurality of the Apparatus Part A. The circuit combines a radio frequency transmitter acting as a modulator 412 for off-on-keying, (OOK) or amplitude shift keying, (ASK) modulation acting as a transmitter for a reference value, command and clock. This circuit comprising the modulator 412, a radio frequency power amplifier 410, the resonant structure described above and plurality of antennas 302 and clock recovery circuits 314 provides reference values, control signals and clock signals to the plurality of the Part A.

In the receive mode, a circuit comprising the radio frequency receiver 413 alternatively acts as an demodulator for off-on-keying, (OOK) or amplitude shift keying, (ASK) modulation acting as a receiver encoded RF modulation in supposition or summing as a response signal from a plurality of Part A.

The permanent digital memory 407, alterable digital memory 408 and digital processor 409 perform a mathematical despreading operation on received encoded RF modulation from 413 in supposition or summing as a response signal from all transmitting Part A. The output of this despreading operation using the a priori stored identification values and encoding code is configured to place the results of the despreading operation combined with a bit weight established by the transmitted reference value into memory corresponding to address locations corresponding to priori stored identification values of the priori stored identification values.

The individual information is filtered and decimated using the digital processor whereby the digital representation of concentration of a chemical or biological analyte acting on the chemoresistive surface of each of Part A. Since the reference value and thus the bit weight can, perhaps, be varied in subsequent operations a complete image of the of concentration of a chemical or biological analyte can be obtained in the manner of a successive approximation converter and stored said in memory.

When the second antenna 403 is tuned to the external radio frequencies, it receives signals from the external radio system 401. The circuit comprises a transmitter and receiver circuit 413 the circuit comprising the permanent digital memory 407, alterable digital memory 408 and digital processor 409 receives an external command from 401 to transmit the set of information from memory corresponding to address locations corresponding to priori stored identification values of Apparatus Part A.

In addition, the visual indicator 414 can be triggered by either the external radio system 401 and receiver circuit 413 or digital processor 409 upon external or internal command.

Thereby using the above, information in memory 407, 408, 414 of the Part B may be transmitted to outside processes, as shown in FIG. 2, upon demand over the external radio link 401 radio link where each set of information concentration of a chemical or biological analyte information, combined with a unique code value to uniquely identify said information from each of the plurality of Part A thereby collecting and providing the temperature, concentration of a chemical or biological analyte data physically inside each carton or item and relaying this data to external information processes inside an electrical dielectric medium of interest thus fulfilling an object of the invention.

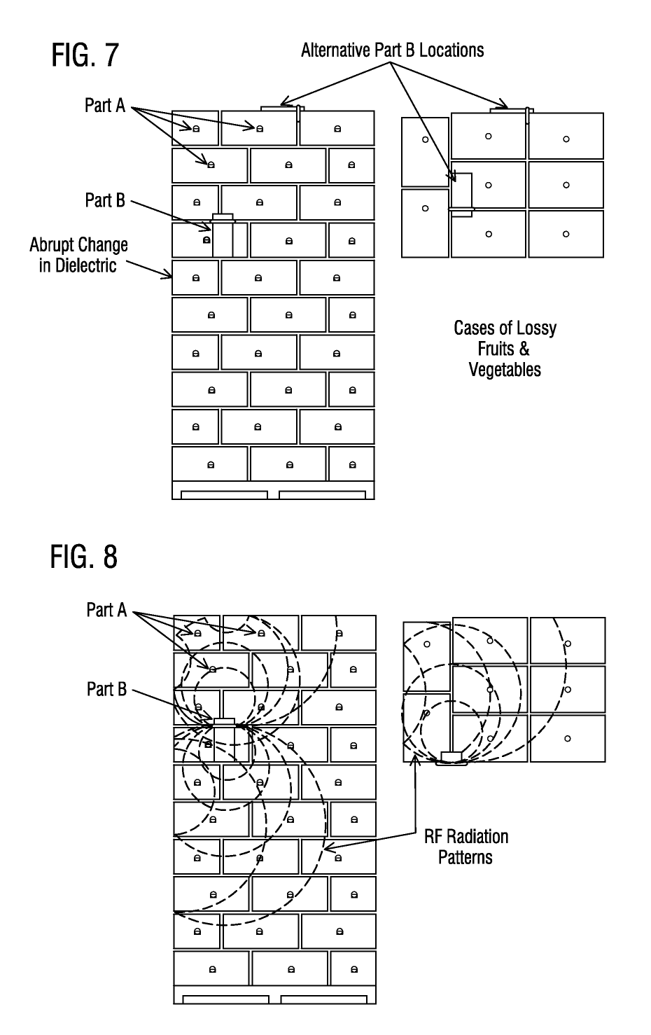

FIG. 7 shows an embodiment of the system, comprising the apparatus, used with a pallet of produce. In an embodiment, a part of the sensor tag apparatus, Part A is placed inside each carton, case or item as warranted as it is packed and placed on a pallet. The chemical, biological and temperature sensors are in direct contact with the fresh food product and are constructed of materials compatible with fresh food products. Part B is attached external to the cartons.

FIG. 8 shows an embodiment of the system with RF radiation patterns. As shown, Part B is in communication with the colony of a plurality of Part A's. The sensor tags, Part A's, measure various physical attributes of the produce.

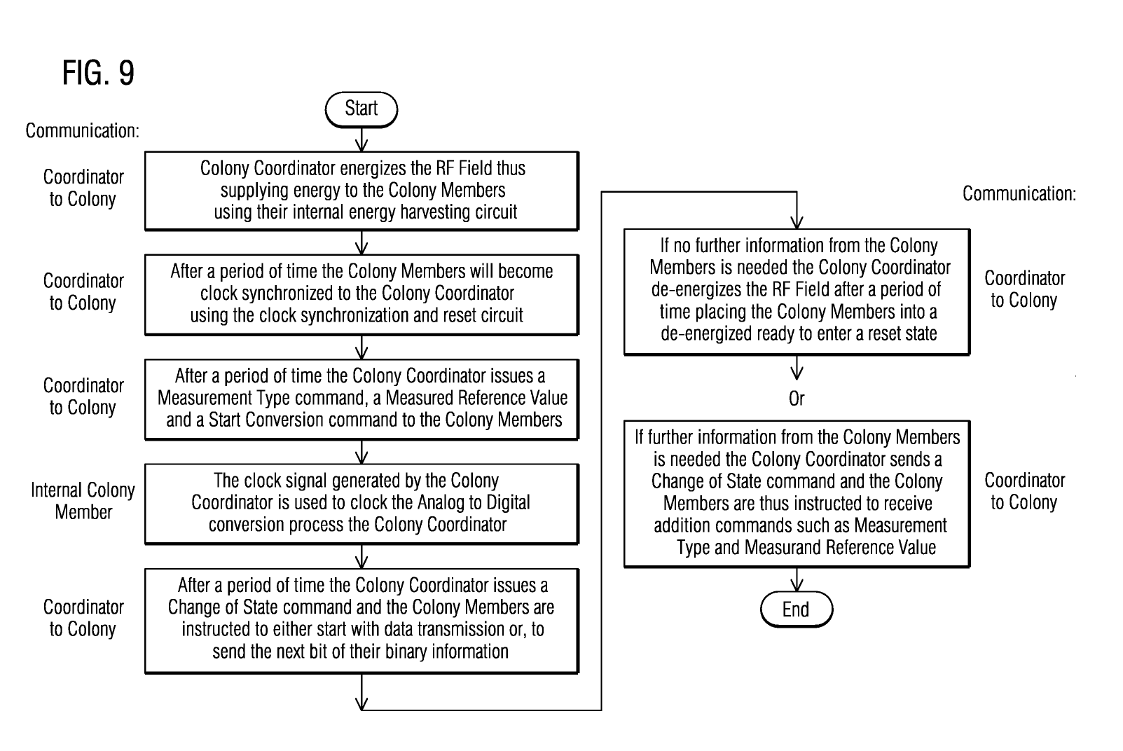

FIG. 9 shows a flowchart of an embodiment of a method. The method may be used for controlling, clocking and synchronizing a number of sensing RFID tags forming a colony inside an electrical dielectric medium of interest for the purpose of communicating data derived thereof acting as radio frequency identification (RFID) tag. As shown, the method may comprise a plurality of first radio frequency identification (RFID) tag apparatus of part A as described herein as colony members with at least one of second radio frequency identification (RFID) tag apparatus of part B as described herein as a colony coordinator. The colony consists of a plurality of first radio frequency identification (RFID) tag apparatus as colony members where the colony members are queried simultaneously and the response of the interrogated tags are collectively clock synchronized by at least one colony coordinator.

The colony members may be controlled by the colony coordinator so that control involves using a synchronization method based on switching on/off the electromagnetic RF field of the apparatus of claim a B acting a colony coordinator tag that powers the Colony member tags by interrupting the wireless power supply generated by electromagnetic RF field of the colony coordinator. In another embodiment, by beginning with transmission by transmitting a modulated start bit in a time slot followed by transmitting the subsequently bit stream in regular sized time slots.

The circuit logic in the colony members clock & reset circuit is tuned to interpret request orders sent from said colony coordinator device based on capacitor discharge behavior and voltage comparator device in the colony members.

Parallel to the circuit logic in the colony members clock & reset circuit is a circuit logic that detects the external clock synchronization request, an internal clock counter in said clock & reset circuit resets the bit index, and start increasing the count when the next intermission is registered so that the colony member tag acquires the actual bit position in the bit sequence in addition to the clock detection circuit a using a memory to preserve the bit index. This memory storage device keeps the storage contents even if the colony tag is not powered for a short term.

The colony coordinator causes an intermission to signal the next time slot for transmission, there before increasing the bit index an additional memory buffer stores temporarily the current bit index and induces the bit transmission, there after increasing the bit index a delayed rewritten in the bit index buffer thereby providing the required delay for stabilized rewriting.

Thereby queries are made to the colony member tags simultaneously and the response of the interrogated colony member tags are collectively clock synchronized and data synchronized by the colony coordinator.

Since the processes begin measured has very low time scales synchronization of the colony members to the colony coordinator will occur after a period of time.

After the synchronization period the colony coordinator issues commands to all colony members at the time, no individual addressing of colony members is required.

The colony coordinator commands issued to the Colony members are at a minimum, Measurement Type, (Temperature or Chemical or Biological measurement), Measurand Reference Value, Start Conversion and Change of State from Reception to Transmission. Optionally a third type of command Resonant Frequency Adjustment may, perhaps be issued.

In the normal sequence of events, the colony coordinator energizes the RF Field thus supplying energy to the colony members using their internal energy harvesting circuit as described above. After a period of time, the colony members will become clock synchronized to the colony coordinator using the clock synchronization and reset circuit as described above. After a period of time, the colony coordinator issues a Measurement Type command, a Measurand Reference Value and a Start Conversion command to the colony members. The clock signal generated by the colony coordinator is used to clock the Analog to Digital conversion process the colony coordinator. After a period of time the colony coordinator issues a Change of State command and the colony members are instructed to either start with data transmission or, to send the next bit of their binary information. If no further information from the colony members is needed the colony coordinator de-energizes the RF Field after a period of time placing the colony members into a de-energized ready to enter a reset state, or, in another embodiment, if further information from the colony members is needed the colony coordinator sends a Change of State command and the colony members are thus instructed to receive addition commands such as Measurement Type and Measurand Reference Value.

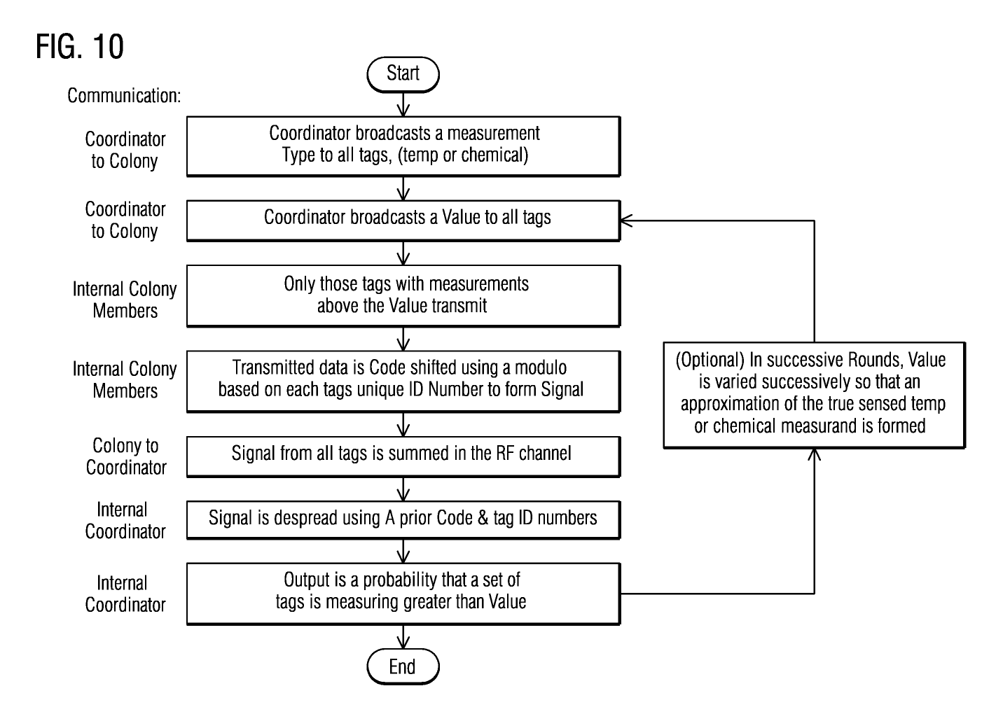

Now referencing FIG. 10, another method in conjunction with the prior method for receiving sensor data from a number of sensing RFID tags forming a colony inside an electrical dielectric medium of interest for the purpose of communicating data derived thereof acting as radio frequency identification (RFID) tag is disclosed. A plurality of first radio frequency identification (RFID) tag apparatus of part A as described above as colony members are provided. At least one of second radio frequency identification (RFID) tag apparatus of part B as described above as colony coordinator is also provided. All colony members comprise a plurality of first radio frequency identification (RFID) tag apparatus as noted above response are synchronized into time slots at the current bit index using the method disclosed in FIG. 9 as described above and the bit information from all colony members composed of a plurality of first radio frequency identification (RFID) tag apparatus as noted above superimposes on the RF channel thereby generating a specific overlaid signal. All colony members are queried simultaneously and the response of the interrogated tags transmit at the same time if and only if the measured value is greater than the reference value transmitted using the FIG. 9 method by the colony coordinator.

In the FIG. 9 method colony members transmit to the colony coordinator using for off-on-keying, (OOK) or amplitude shift keying, (ASK) modulation in supposition or summing as a response signal from a plurality of colony members to the colony coordinator. The colony coordinator performs a mathematical despreading operation on said received encoded RF modulation in supposition or summing as a response signal from a plurality of colony members using both a priori stored identification values and encoding values wherein the following steps are repeated on the plurality of colony members. The colony coordinator calculates a probability that an individual colony member has responded indicating that a particular colony member senses a measurement value higher than the reference value transmitted by the colony coordinator during the operation of the FIG. 9 method described above as graphically illustrated in FIG. 9A, tags #1 to #n. The colony coordinator places the results of the despreading operation into an individual memory location reserved for the specific a priori stored identification value for that specific colony member tags #1 to #n. The colony coordinator calculates a combined with a bit weight established by the transmitted reference value from the FIG. 9 method described above and the probability that an individual colony member has responded indicating that individual colony member senses a measurement value higher than the reference value and places the results of the bit-weight operation into an individual memory location reserved for the specific a priori stored identification value for that specific colony member tags #1 to #n. The colony coordinator performs filtering and decimation operations on the individual colony member combined bit weights in memory whereby the digital representation of said concentration of a chemical or biological analyte acting on the chemoresistive functionalized surface of each sensor or temperature of that specific colony member tags #1 to #n.

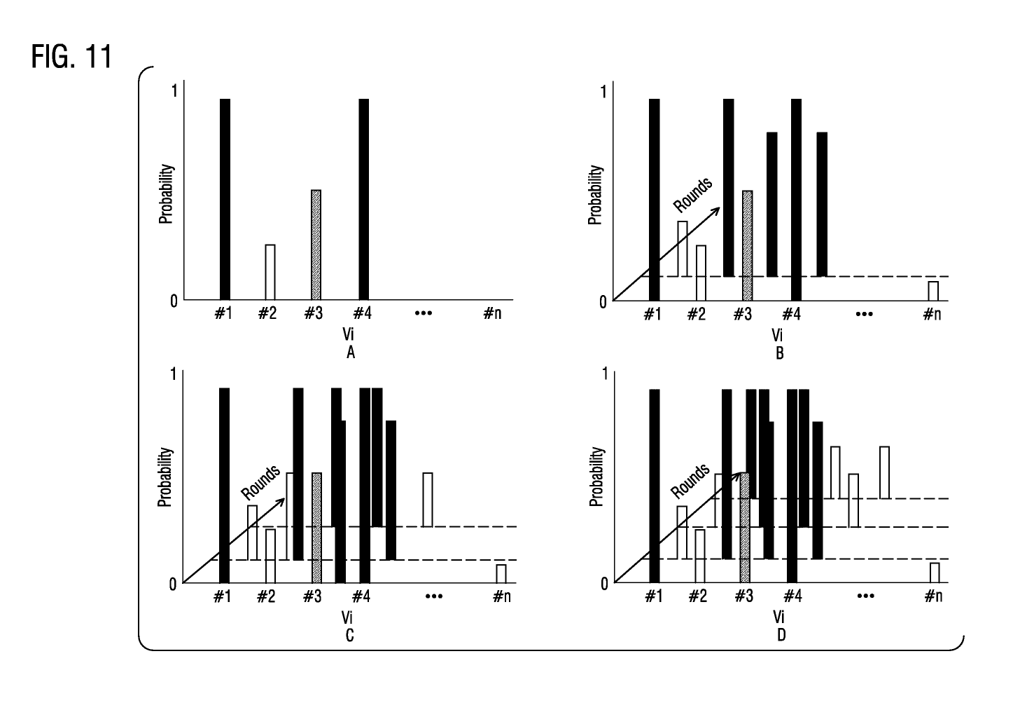

The colony coordinator may, perhaps vary the Reference Values using the FIG. 9 method described above to obtain in the manner of a successive approximation converter a variable resolution ensemble of the digital representation of the concentration of a chemical or biological analyte acting on said functionalized surface of each sensor or temperature of that specific colony member tags #1 to #n, wherein said ensemble is stored in memory as graphically illustrated in FIGS. 11. B, C and D.

As a non-limiting example as graphically illustrated in FIG. 11D by applying appropriate bit-weights Tag #1 would read binary 1001, Tag #2 would read binary 0001, Tag #3 would read binary X110, and Tag #4 would read binary 1010 where X denotes an uncertain value. Thereby the combination of the two method illustrated in FIGS. 9 and 10 query the colony of tags simultaneously and the digital representation of a concentration of a chemical or biological analyte acting on said chemoresistive functionalized surface of each sensor or temperature of the interrogated colony member tags are collectively obtained and providing the temperature, concentration of a chemical or biological analyte data physically inside each carton or item and relaying this data to external information processes inside an electrical dielectric medium of interest thus fulfilling the object of the invention.

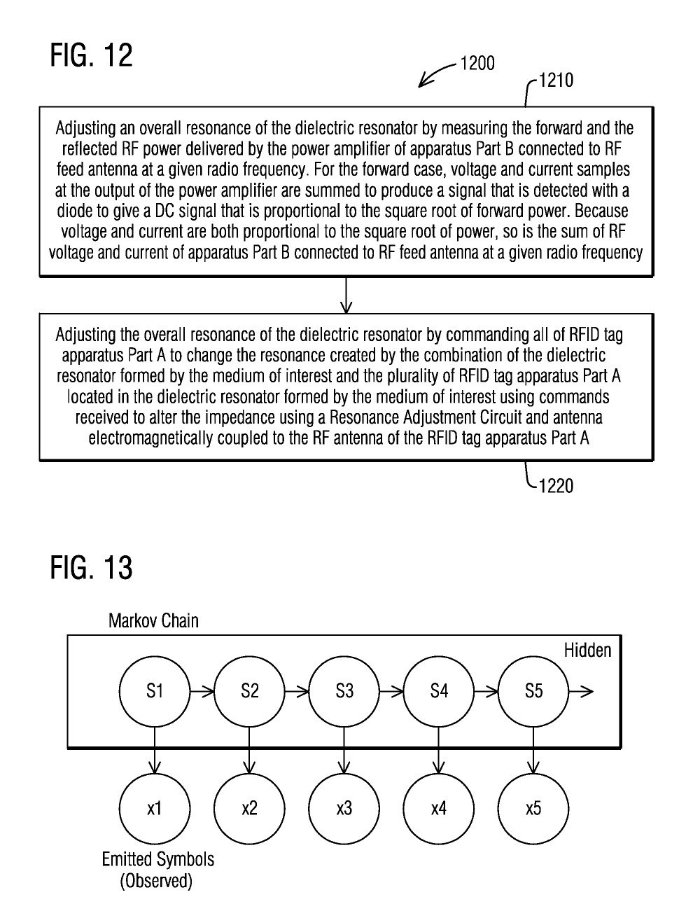

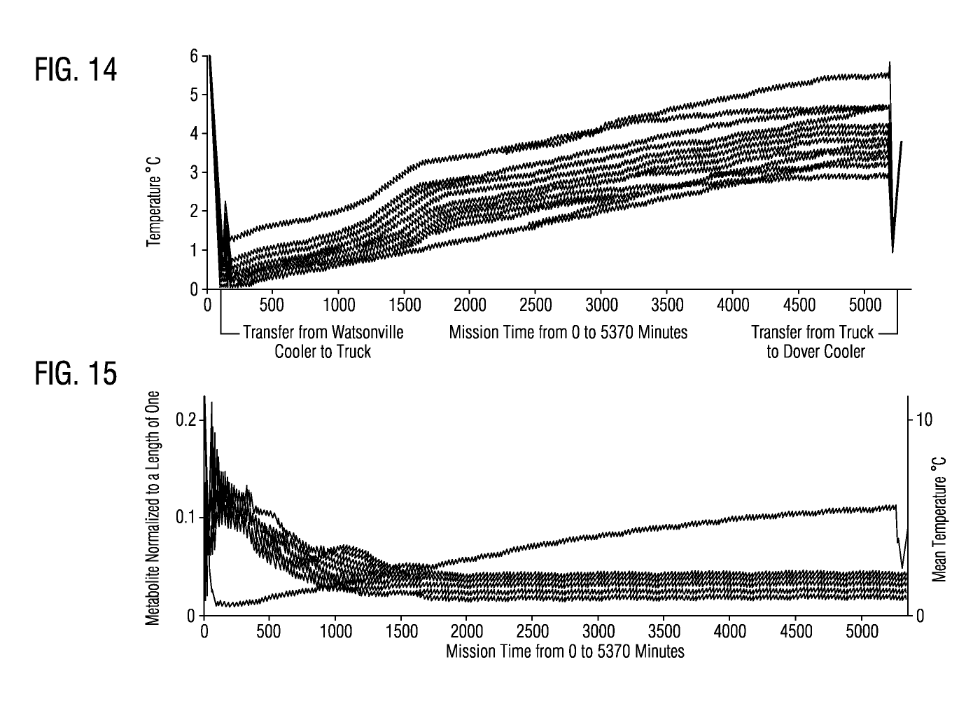

A third method 1200 illustrated in FIG. 12, for optimizing the resonance of each of the plurality of RFID tag apparatus Part A as described above containing one of these resonators consisting of an inductive coil and a capacitor whereby the resonance point inside an electrical dielectric medium of interest excited by a feed circuit of RFID tag apparatus Part B to optimally harvest power from the RF feed and communicate with the RF feed circuit. The Third Method consists of two parts. A first part involves adjusting an overall resonance of the dielectric resonator by measuring the forward and the reflected RF power delivered by the power amplifier 410 of apparatus Part B connected to RF feed antenna 402 at a given radio frequency. For the forward case, voltage and current samples at the output of the power amplifier 410 are summed to produce a signal that is detected with a diode to give a dc signal that is proportional to the square root of forward power. Because voltage and current are both proportional to the square root of power, so is the sum of RF voltage and current of apparatus Part B connected to RF feed antenna 402 at a given radio frequency, at 1210.

For the reflected power, a current sample 180° out of phase with the actual current being detected is required. For reflected power, voltage and current are sampled at the Resonant adjustment circuit 801 and antenna 802 electromagnetically coupled to the RF feed antenna 402 and summed to produce a signal that is detected with a diode to give a dc signal that is proportional to the square root of reflected power.

For a given load impedance, the method iteratively changes the electrical characteristics of the resonant adjustment circuit 801 until impedance matching is achieved within a margin of error wherein the voltage and current samples present nearly equal amplitudes at the summation point and are nearly exactly out of phase with each other within a tolerance, giving a very small or zero summation signal. This is also peak-detected to give a dc signal proportional to the square root of reflected power.

It will be appreciated to those skilled in the art that the actual load impedance at power amplifier 410 is the combination of its own impedance, the impedance of the RF feed antenna 402, the combination of the Resonant adjustment circuit 801 and antenna 802 and most importantly the combination of the dielectric resonator formed by the medium of interest and the plurality of RFID tag apparatus Part A located in the dielectric resonator formed by the medium of interest.

The first part of the method shown in FIG. 12 iterates until no changes in the ratio of the forward and the reflected RF is achieved.

Subsequently, a second step 1220 of the method 1200 provides for adjusting the overall resonance of the dielectric resonator by commanding all of RFID tag apparatus Part A to change the resonance created by the combination of the dielectric resonator formed by the medium of interest and the plurality of RFID tag apparatus Part A located in the dielectric resonator formed by the medium of interest using commands received to alter the impedance using a Resonance Adjustment Circuit 703 and antenna 702 electromagnetically coupled to the RF antenna 302 of the RFID tag apparatus Part A.

The second step 1220 iteratively changes the electrical characteristics of the resonant adjustment circuit 703 and antenna 702 electromagnetically coupled to antenna 302 using commands from RFID tag apparatus Part B until impedance matching is achieved by measuring the forward and the reflected RF power delivered by the power amplifier 410 of apparatus Part B connected to RF feed antenna 402 at a given radio frequency as described above in the first step 1210 of the method 1200 where by adjustments Resonant adjustment circuit 801 and antenna 802 are not made.

It will be appreciated to those skilled in the art that the actual load impedance at power amplifier 410 is the combination of its own impedance, the impedance of the RF feed antenna 402, the combination of the Resonant adjustment circuit 801 and antenna 802 and most importantly the combination of the dielectric resonator formed by the medium of interest and the plurality of RFID tag apparatus Part A located in the dielectric resonator formed by the medium of interest and the resonant adjustment circuit 703 and antenna 702 electromagnetically coupled to antenna 302.

The second step 1220 iterates until no changes in the ratio of the forward and the reflected RF is achieved.

Thereby both steps 1210, 1220 maximizes the resonance of the dielectric resonator formed by the medium of interest to optimize the power transfer and communications to the plurality of RFID tag apparatus Part A located in the dielectric resonator formed by the medium of interest is achieved.

The system shown in FIG. 2 whereby may organize a great plurality of first radio frequency identification (RFID) tag apparatus of part A as described above as colony members as a subset of a plurality of second radio frequency identification (RFID) tag apparatus of part B as described above as colony coordinator as a set utilizing the method of FIG. 10 and the method of FIG. 11 each reporting chemical, biological and temperature information into a colony whereby said system decodes said chemical and biological information and processes said information from each individual colony members sensor tags is a member utilizing a known code sequence to form an electronic digital domain representation of said chemical, biological and temperature information.

Thus, as disclosed, the system may comprise a plurality of first radio frequency identification (RFID) tag apparatus of part A as described above as colony members, and at least one of second radio frequency identification (RFID) tag apparatus. A label reading device may be connected to a first mobile device apparatus, said device is connected to the internet. An unknown number of other second mobile device and fixed apparatus, said devices are connected to the internet A computer is provided as an analysis element connected to the internet.

The system formats both the set and subsets of decoded chemical and biological information thereof for relay to a gateway element for either immediate or delayed retransmission to the computer analysis element. The system contains a computer analysis element performing computations on said information to estimate the amount of Chemical and Biological analyte and temperature characteristics sensed by each individual member of each colony. The system 100 may also organize an even greater plurality of sensor tags each reporting chemical, biological and temperature information into many colonies whereby said system obtains data from thousands of colony member tags thereby harvested by a plurality of colony coordinators and further the data from thousands of colonies whereby the system organizes groups of these colonies each reporting chemical, biological and temperature information into groups of colonies whereby the system decodes the temperature chemical and biological information and processes the information from each of the colony member sensor tags in a time correlated database to form an electronic digital domain representation of said chemical, biological and temperature information for further analysis. said system formats the time series database based on the interpreted chemical and biological information for relay back to a decision point thus creating actionable intelligence of decay at the carton and item level.

In the preferred embodiment the application is toward shipping carton and item level tagging e.g., where a plurality of said cartons and perhaps, Items are equipped with colony members sensor tags apparatus of part A as described above incorporating a method described above combined with a single a single signal processing bridge apparatus of part B managing the colony as colony coordinator mounted on a shipping pallet whereby said bridge apparatus of part B decodes and converts said information from the members of the Colony to standard digital formats to both sense and relay said Chemical & Biological decay information at the shipping carton level back to a decision point thus creating actionable intelligence of decay at the carton and item level.

Embodiments disclosed herein are based on a Hidden Markov model (HMM) for the assessment of the deterioration of the product. The HMM is a statistical model in which the system being modeled is assumed to be a Markov process with unobserved (hidden) states. Hidden Markov models (HMM) are based on the estimation of a transition matrix which states the probability that the assessment of a product changes from one category to another. They also include conditional probabilities that reflect random errors in the assessment.

Functionally a succession of states representing an evolution of a quality of each product at the carton level is obtained. An estimate of a probability of assignment of each food carton to a correct state is performed. Then a calculation of transition probabilities between different states of the product (within it housing, such as, but not limited to a carton) is performed.

In a Hidden Markov model, illustrated in FIG. 11, the state is not directly visible, but the output, dependent on the state, is visible, (measurable). Advantages of the embodiments disclosed herein include, but are not limited to providing a simpler, a single model to accommodate all cultivars of the product because HMM does not require a priori definition of relevant biological processes to be effective and it becomes more accurate with the accumulation of experience. Another non-limiting benefit is data compression is utilized wherein the smaller HMM estimate of the succession of states representing the evolution of the product quality is itself the target data set rather than large time series data sets. Also, use of HMM estimates of the succession of states is by relational, thus allowing analysis by familiar tools.

When produce is the product, each individual produce has its own chemical state-space representation, a mathematical model of the physical system as a set of input, output and state variables related by first-order differential equations. State Space refers to the space whose axes are the state variables. The state of the berry as a chemical system can be represented as a vector within that space.

Input States are measurable values such as temperature and humidity and Output States are primarily volatile organic compounds (VOC's) and heat. State Variables are based on a rate of reaction, (this may be different than Respiratory Quotient (RQ) of the respiratory metabolism. This process of respiration involves combining O2 in the air with organic molecules in the tissue (usually a sugar) to form various VOC compounds and eventually CO2 and water. Closely coupled but an independent state is the energy produced by the series of reactions making up respiration. This energy can be captured as high-energy bonds in compounds used by the cell in subsequent reactions, or it can be lost as heat. The energy and organic molecules produced during respiration are used by other metabolic processes to maintain the health of the commodity. (Heat produced during respiration is also known as “vital heat,” and it contributes to the refrigeration load that must be considered in designing storage areas.)

Because it is currently impractical to instrument each individual produce in a carton or another housing embodiment herein apply an assumption that closely located product (in a particular container) create an ensemble that is reasonably constant in geometry and mass so that individual product state-vector distributions create a Gaussian state-vector or ensemble state-vector for the product in the housing and, related ensembles of produce state-vector distributions create an ensemble state vector for a larger collection of packages, such as, but not limited to a pallet. As time passes and more state observations are assimilated, the distributions become closer to Gaussian, even if the initial ones are clearly non-Gaussian. Then, since State Variables are many and are uncontrolled variables, such as, but not limited to weather, planting time, cultivar etc., the data analysis for dynamic degradation is based on a Markovian model and the use of Hidden Markov Modeling, (“HMM”), as disclosed above, to generate pseudo State Variables for the ensemble. An absolute value of each measurement is not important. Instead, what is important is the trajectory of the State Vector.

FIG. 12 is a graph illustrating temperature over a mission time. As shown, using embodiments disclosed herein, temperature is accurately tracked where drastic changes are able to be identified based on a time during a mission (transport time).

FIG. 13 is a graph showing metabolite data during a mission. Specific, metabolite is normalized to a length of one over a length of the mission. The mean temperature is also shown over the length of the mission.

Thereby using the above, apparatus, method and system collecting data from inside an electrical dielectric medium of interest for the temperature, concentration of a chemical or biological analyte located physically inside each carton or item and relaying this data to external information processes the object of the invention is thus fulfilled.

In the Apparatus Part A as described above the potentiostatic measurement circuit 306 can be replaced with a galvanometric measurement circuit. In the Apparatus Part B as described above the battery 404 and second radio external radio system 413 and antenna 403 tuned to a second radio frequency can be replaced with a wired interface circuit to an external interface.

It will be appreciated to those skilled in the art that if the first electrical dielectric medium of interest has an electromagnetic wave number less than three times the second dielectric medium of interest the inventive System, Method and apparatus will function as described above if bounded by an electrical conductor.

In a further example embodiment: many commodities can become damaged by water onboard ships holds. This can take many forms but the most common involve either ingress of water from an external source (sea water or rain water) or movement of moisture within the hold leading to cargo damage. Often more than one of these mechanisms may be in place and it becomes important to understand what processes cause which phenomena and which is likely to be more significant for proper remediation.

Grain, fertilizer, coal, and other commodities primarily move around the world in what are called dry bulk vessels. Dry-bulk is a term used to describe ocean going vessels that have 4-9 cargo holds into which coal, ore, metals, fertilizer, and grains can be directly poured into and easily discharged in bulk. These vessels are configured differently than general cargo (tween deck vessels), tanker, liquid bulk, and container ships. The world dry-bulk fleet is comprised of various cargo size vessels. The larger ships, from Suezmax and up, are not typically used to carry grains and oil seeds. The focus of this embodiment will be on agricultural-type commodities such as grain and animal feedstuffs, but some of the principles will apply equally to other cargoes,

It is the focus of this embodiment to configure the apparatus Part A as described above with at least one first sensor comprising an area of a reversible chemoresistive surface VR 305 is made sensitive to moisture and coupled to a potentiostatic measurement circuit 306. The temperature sensor is not changed.

In this embodiment of the invention a plurality of the apparatus Part A as described above is placed inside in the material, for example wheat, as it is packed the hold of the bulk carrier. From a RF perspective the most significant difference from a pallet of fresh food in the preferred embodiment is that instead of relying on the step change in dielectric permittivity from a material with a high permittivity to low permittivity to reflect and distribute EM radiation inside a pallet, a Bulk Carrier carrying wheat has a low dielectric permittivity, wheat, bounded by a good conductor, the steel structure of the ship that none the less can form a resonator possible to excite the resonators used in the colony members to both provide power and communicate with the colony coordinators, one per hold.

In this embodiment the feed 402 used in the apparatus Part B, colony coordinators as described above are used to excite a dielectric resonator formed by the wheat/steel hull combination per hold to reflect and distribute EM radiation inside the hold. In some instances, perhaps, it may be desirable to replace the second radio system 403 and 401 with a wired interface and the battery 406 with ships interfaces and power.

This embodiment can be used to detect condensation (ship's sweat) wetting of bulk cargoes which is essentially a surface phenomenon and rarely penetrates more than a few centimeters into a stow. It happens when the steelwork is cooled by external conditions leading to moisture deposition on the cold steel.

This moisture migration is a more complex phenomenon and is usually caused by temperature differences. Moisture tends to move from warmer cargo into cooler parts of the stow. Large movements of moisture only take place when there are large temperature differences affecting large areas. Usually moisture migration involves moisture being driven upwards in the stow by warm cargo.

It is important therefore to see moisture migration as the consequence of a temperature gradient, not an actual mechanism causing damage in its own right. Many attribute damage associated with caking or moldiness' to “moisture migration” without actually considering what has caused the moisture movement.

In some cases, cargo can become cooled by external conditions, and this may result in moisture migrating to that cooler cargo which then deteriorates. This only affects cargo at or very near the periphery of a stow.

More usually, moisture migration is caused when cargo becomes warmed. This can take place by heat transfer from an external source such as a bunker tank. The amount of bulk cargo which can be damaged by an external heating source tends to be limited and restricted to that adjacent to the source, but there can be some spread of damage away from the immediate area by moisture migration. It is unusual, however, to see this spread far into the stow.

Another possible reason for cargo becoming warm is when it self-heats as a consequence of its inherent moisture and temperature. This can result in large regions of a bulk cargo becoming warm and caked, and large migration of moisture can result from this. This, in turn, will tend to cause condensation and wetting at the surface of the stow, but it is important to note that it is the self-heating which started the process and is the underlying cause behind all of the observed deterioration. Self-heating is the fundamental process driving “moisture migration” in nearly all instances.

By embedding the colony members in the bulk cargo this embodiment can be used to detect this moisture a shipper will be able to understand exactly what the condition of the bulk cargo is during transit with a granularity not previously possible.

Further, shippers will be able to understand exactly what the condition of the bulk cargo is during transit. Using the system shippers can dynamically match bulk destination and distribution routing with relative shelf life expectancy to ensure delivered product freshness to make meaningful decisions about a particular ship load.

Therefore, an object of the invention using a and moisture and temperature sensor to monitor and report the severity of moisture in bulk cargos such as grains utilizing the System combined with ships wireless reporting network, and possibly, algorithms for processing the signals being output by the sensors, Methods and Apparatus described above thus creating an RFID tag of the type described above used advantageously to make a moisture and temperature detecting solution to reduce Ocean Cargo Insurance Premiums is realized.

In a further example embodiment: while the worldwide pharmaceutical market is in excess of $400 billion, the U.S. pharmaceutical market is valued at greater than $200 billion and includes about 14,000 drugs. To reduce the costs of producing these drugs, U.S. manufacturers are going abroad. However, this raises counterfeit concerns. According to Physician's News Digest, “as drug manufacturers have moved their production plants worldwide, some countries like Pakistan, India, Thailand and Mexico have become havens for the production of unapproved, knock-off drugs.”

About 32 drugs are the most commonly counterfeited and or adulterated due to their high dollar value and physical characteristics. Because many of these pharmaceuticals come in liquid form, counterfeiters can easily dilute them with water.

This is a chemical problem requiring a chemical anti-counterfeiting solution to secure the chain of custody per the Prescription Drug Marketing Act (PDMA) to maintain product ‘pedigrees’.

In this embodiment described above, it is combined an algorithm using the temperature dependence of reaction rates to detect counterfeiting and adulteration. In the embodiment, a set of 16 different inert carrier tagging solutions are available that slowly decay over time into measurable but safe reaction products. Zero to four from this set of tagging solutions are included in the drug at varying starting concentration or other such data deemed appropriates to introduce uncertainty for the counterfeiter. The method and apparatus described above is included inside the package in contact with the drug at the time that the drug is first packaged. The method and apparatus described above has a unique identification number, temperature-sensing element with temperature logging memory and a four measure and chemical sensor tailored to the specific tagging solutions employed.

At any impact point in the delivery chain the method and apparatus described above can be read using mobile readers and the results linked back to a cloud based Anti-Counterfeiting Analyzer platform. The cloud based Anti-Counterfeiting Analyzer platform compares the encrypted ID number that is referenced back to the specific tagging solution set and starting concentration or other such data deemed appropriates, the time and the temperature log to form an mathematical inverse estimate of the expected decay products based on the algorithm. If this estimate is in variance with measured data and a history the product will be flagged and these results communicated to the impact point in the supply chain for action.

The drug production plants are blind to the exact inert carrier tagging solutions and concentration or other such data deemed appropriates actually employed on a lot-by-lot basis eliminating production plant shrinkage. The introduction point of an adulterant can be determined by reverse tracing the steps of supply chain.

This technology is automatic and is much less costly than lot sampling using expensive lab instruments and skilled labor. The tags can be made at a price point allowing disposable tags. Many current and costly detection processes could be refined or even discarded though the use of the invention at the point of impact to maintain product ‘pedigrees’.

The system has deliberate blind spots built in for security. In the envisioned Concept of Operation, ConOps, tags using the method and apparatus described above the will interact with a smart phone app though a common carrier onto the internet. The interrogator or smart phone app are blind to what is being sensed.

The ID number, the raw sensed analyte and time stamp data will be transmitted over these links to a secure List Server that cross reference the ID number and forward the identity of the chemical and starting concentration or other such data deemed appropriate used in that tag to another secure Analyzing Server. The List Server is also blind to what is being sensed.

The Analyzing Server applies the algorithm with the time stamp and actual identity of the chemical and starting concentration or other such data deemed appropriate to determine the tags current values that are compared to the tags supply chain history to authenticate the tag. The Analyzing Server then forwards the status authentication status to an Authentication Server for action. The Analyzing Server is blind to what action if any is performed by the Authentication Server.

It is not our place to tell the Drug manufacturer what to do with this information at this point, though the Authentication Server could alert the smart phone app if desired.

Therefore an object of the invention using a chemical anti-counterfeiting solution to secure the chain of custody per the Prescription Drug Marketing Act (PDMA) to maintain product ‘pedigrees’ utilizing the system, method and apparatus described above combined an algorithm using the temperature dependence of reaction rates to detect counterfeiting and adulteration, creating an RFID tag of the type described above used advantageously to make a chemical anti-counterfeiting solution to secure the chain of custody by sensors (each carrying a selective area toward a different chemical species) combined into a wireless sensor grouped on an isolating housing provided with an appropriate wireless network, and possibly, circuitry and devices for processing the signals being output by the sensors is realized.

In yet another example embodiment, the FDA is overhauling the food safety system, and companies impacted by the FSMA ruling will be forced to abide by these eventual new regulations. At this time, it is still unknown how soon shippers, drivers, carriers, and receivers of food products must comply. The FDA may seemingly be dragging its feet with regard to the proposed FSMA ruling, since it is immersed in a large undertaking. In spite of the fact that additional time is needed to initiate this enormous ruling, the department is fully intent on enforcing these sanitary and food safety rules in 2015 and early 2016. The FDA is already focusing inspections on companies that deal in food handling, storage, shipping, and sanitation. It will target certain processes within a facility that are most likely to be vulnerable, rather than targeting specific foods or hazards. From the FDA analysis:¹ ¹Docket No. FDA-2013-N-1425

The Intentional Adulteration Group was a ‘subset’ of the 14,000 companies impacted, and it was comprised of 4,624 companies. This number was calculated by conducting an impact analysis using economics based upon companies doing more than 10 million annually in sales. The analysis was taken using SIC codes and Dunn and Bradstreet reports.

In essence, there will be an expectation placed upon every vendor, shipper, supplier, and receiver of foods to comply with these standards. Companies will need to be ready with records to verify they are in compliance. Facilities will sometimes be required to provide this proof within 24 hours. In the context of ‘preventable control,’ the FSMA ruling gives the FDA legal authority to access records, determine reasonable belief the food might be adulterated, and allow 3rd parties to help enforce these rules.

Therefore, an object of the invention advantageously using the utilizing the system, method and apparatus described above designed to target the specific food product itself using the temperature dependence of reaction rates and history to detect spoilage, creating an RFID tag of the type described above using advantageously RF technology, wherein several sensors (each carrying a selective area toward a different chemical species) combined into a wireless sensor grouped on an isolating housing and orientation provided with an appropriate wireless network, and possibly, circuitry and devices for processing the signals being output by the sensors is realized.

In a still further example embodiment: falsified medicines are a growing risk worldwide. Twenty-four percent of counterfeit products seized at EU borders are medications. To keep them out of the legal distribution chain in Germany, pharmaceutical manufacturers and wholesalers as well as pharmacists in Germany are joining forces to offer a better means of preventing falsified medicines from being handed out—by developing a security system that will be able to test whether medicinal products are genuine. The securPharm initiative launched by their associations will elaborate this system and test it in a pilot in 2013. The system is intended to comply with the EU's new stipulations for preventing counterfeits and—after wide-scale rollout—ensure that patients still have a safe source for their medicines at all times.

Medications that may have been falsified must therefore be furnished with a security feature that can be used to check the authenticity of the medication at any time along the entire supply chain from when it leaves the production facility right through until it is dispensed to the end user. 750 million packages of prescription medications are dispensed every year in Germany alone.

Currently securPharm has developed a special coding system for Germany based on Data Matrix codes in order to continue to guarantee a high level of security in view of the increasing level of counterfeiting around the globe in order to maintain the legal supply chain from the manufacturer to the wholesaler to the pharmacy. In this technology the product and serial number is displayed on the packaging of the medication. This code can be used to verify authenticity at every point along the legal supply chain. According to the end-to-end approach, verification must be carried out in a pharmacy before the medication is dispensed.

Weakness; securPharm only tracks the pharma package not the pharma itself so it is still vulnerable to tampering including theft, shrinkage, (reducing the amount of pharma in the package), and replacement with counterfeits and adulteration for sale outside of the EU.

In this embodiment the method and apparatus described above is included inside the package combined with a known gas sealed inside the package at the time that the drug is first packaged.

The System Method and Apparatus described above has a unique identification number, may possibly, include a temperature-sensing element with temperature logging memory and a single measurand chemical sensor tailored to the specific gas employed. The pharma packaging is designed to release the gas if the packaging is tampered with thereby becoming diluted with room air.

At any impact point in the delivery chain the method and apparatus described above can be read using RF readers to detect the introduction of room air. If room air is detected, the product will be flagged and these results communicated to the impact point in the supply chain for action.

The introduction point of tampering can be determined by reverse tracing the steps of supply chain.

This technology is automatic and is much more effective than a simple bar code in detecting theft, shrinkage, (reducing the amount of pharma in the package), replacement with counterfeits and adulteration. The tags can be made at a price point allowing disposable tags. Many current and costly detection processes could be refined or even discarded though the use of the invention at the point of impact to maintain product ‘pedigrees’.

It is not our place to tell the Drug manufacturer what to do with this information at this point, though the system could alert the smart phone app if desired.

Therefore, a further object of the invention for the detection of tampering to maintain product ‘pedigrees’ utilizing the system method and apparatus described above to detect the introduction of room air, creating an RFID tag of the type described above used advantageously to make a chemical anti-counterfeiting solution to secure the chain of custody by sensors (each carrying a selective area toward a different chemical species) combined into a wireless sensor grouped on an isolating housing provided with an appropriate wireless network, and possibly, circuitry and devices for processing the signals being output by the sensors is realized.

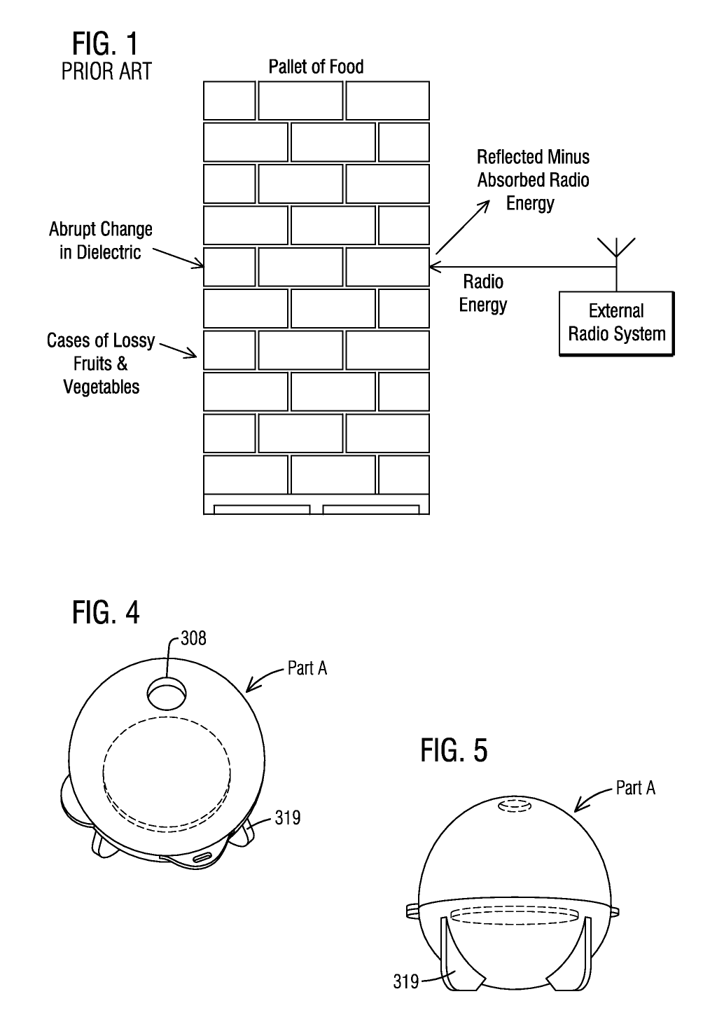

The physical wireless layer of the invention takes advantage of the fact that an Electromagnetic, (EM), wave incident on the surface of a dielectric material can either be reflected (i.e. reflected wave) or be transmitted into a material (i.e. transmitted wave). When there is a shift between dielectric properties going from air, (free space), to another material the transmitted wave will be refracted or bent away from its original path. This effect is commonly observed in a glass of water using light, and is known as Snell's Law. The refracted wave is gradually attenuated as it is converted to thermal energy, with exponent proportional to the imaginary components of the dielectric permittivity. All conventional RFID techniques are limited by the abrupt dielectric shift between air and a fresh food product when attempting to transmit a signal from the outside of a pallet of fresh food and penetrate to the inside as illustrated in FIG. 1. “Conventional” is used to refer to standards as established by either the International Organization for Standardization (“ISO”) or IEC standards.

Turning back to FIGS. 7 and 8, if EM waves are introduced inside of a dielectric material with a higher permittivity from a transmitter circuit and there is a sufficient dielectric shift going to another material with a lower permittivity, air for example, the EM waves are internally reflected and bounce back and forth within the higher dielectric materials physical boundaries.

This technique will be recognized to those skilled in the art as commonly employed at microwave frequencies where the dimensions of the dielectric material are controlled a high quality tuned resonator circuit can be created using a RF feed. In addition, if the walls of the dielectric material are partially transparent to EM waves, radio power will radiate outward forming EM radiation patterns that can be constructed to match any conventional resonant wire type or patch antenna.

Those skilled in the art will recognize that utilizing appropriate scaling rules this same technique works equally well for lower frequencies.

The key to the inventive concept is the recognition that a pallet of fresh food can be viewed as a composite RF material and formed into a lossy dielectric resonator rather than a single monolithic block. Of great importance to the invention are the Effective medium approximations or effective medium theory (sometimes abbreviated as EMA or EMT) which pertains to analytical or theoretical modeling that describes the macroscopic properties of composite materials.

In the specific area of fresh food on a pallet an inhomogeneous composite is incidentally created, made up of lossy fruits, vegetables or meat and nearly lossless air surrounding them in the typical packaging carton. To aid cooling of the food during transport the volume fraction in fruits, vegetables or meat packaging is always designed to be 50% so as a composite the effective dielectric losses are always about 60% lower than the individual constituent values of lossy fruits, vegetables or meat. The invention goes a step further placing a third type of inclusion in the form of electromagnetic resonators embedded in the fresh food pallet composite.

Each of the plurality of sensor tag apparatus in the invention contains one of these resonators consisting of an inductive coil and a capacitor. Theoretically it is possible to design these resonators so that at a single frequency the food pallet composite is transparent to EM radiation. However, the invention uses these resonators to both harvest power from the RF feed and communicate with the RF feed circuit. Part of the invention is a method used to “tune” these circuits. Because all RF phase information is lost the inventive method also incorporates the limitation in the communication protocol.

In an embodiment, a part of the sensor tag apparatus is placed inside each carton, case or item as warranted as it is packed and placed on a pallet. The chemical, biological and temperature sensors are in direct contact with the fresh food product and are constructed of materials compatible with fresh food products as illustrated in FIG. 3.

In addition, the housing of the sensor tag apparatus of the inventive concept functions to prevent mechanical crushing forces during shipping, provide a known dielectric in the reactive electromagnetic field of its antenna and maintain correct orientation of the antenna under mechanical vibrations typical of shipping.

It is not desirable for the pallet of fresh food to radiate beyond the pallet because this wastes RF power and can cause interference with other nearby pallets therefore the second apparatus in the invention incorporates an RF feed designed to limit such EM radiation leakage while distributing EM energy as uniformly as possible inside the pallet. The design of this RF feed allows it to be easily relocated to the optimal feed point for a particular pallet configuration and food type.

If a single feed is not sufficient to distribute EM energy as uniformly as required inside the pallet dielectric multiple RF feeds can be added as “clones” of the original.

In the invention, the plurality of sensor tags apparatus can be thought of as a members of a colony where these sensor tags measure various physical entities as illustrated in FIG. 8.

These colony members are connected wirelessly with a single processing function inside a battery operated apparatus acting as a colony coordinator equipped with the RF feed device.

At least one colony coordinator apparatus is required per pallet but any number can be used to achieve complete EM coverage of the pallet.

The colony member apparatus cannot function without a colony coordinator because they have no internal energy source.

The colony coordinator is also equipped with a second antenna used to relay stored sensor data from colony members to the internet cloud using high-speed standard RF signals on demand as illustrated in FIG. 1.

In principle, the colony coordinator could query colony member tags individually using any of the well-established protocols. However, implementing these protocols is not feasible since the sensor tags apparatus need to be simple for low cost allowing disposability. This communication protocol is specifically designed to match chemical processes rates of change.

Unlike conventional RFID which uses TDMA and complex tag arbitration logic the inventive method uses a much simpler version of Code Division Multiple Access, CDMA, to communicate from the many colony member tags located inside each carton to a single colony coordinator mounted to the pallet as shown in FIG. 3.

Unlike conventional RFID the inventive method is designed to use no on tag arbitration in order to simplify the colony member tags apparatus for low costs allowing disposability.

The colony coordinator is programmed with each colony member's ID number as part of a binding operation when the pallet is originally built up. The colony member ID is used to create a code where the inverse of the received data it is an encrypted image of the ID number. Only colony members with a sensed value exceeding a reference value transmitted by colony coordinator reply with the encoded ID number, not the measured value itself. The RF signal from each colony member is summed in the RF channel with the other replying members. In the colony coordinator this sum is despread in a mathematical operation with a processor using the known colony member's ID number to produce a set of probabilities of which colony members are in fact replying. This set of probabilities is correlated with the reference values so that a sensor bit weight can be assigned to the set of colony members under the control of the colony coordinator. The sensed bit weight is stored in memory as a “virtual tag” per that colony members ID number.

In the Method the reference values can be changed in successive interrogation rounds in order to form a successive approximation analog to digital converter. This technique works because the input data is very slow matching chemical processes. It has the advantages that the BER is excellent in AGWN & IN, is power efficient and has built-in encryption. The converter resolution is variable and the weighted ensemble is very accurate and also Coherent due to the a priori knowledge of the reference values, ID numbers and encoding values, as well as Stochastic due to the nature of the processes themselves.

It is important to note the exact value from each measurement is not recovered using this method, rather a statistically related data set for each measurement value is recovered. This is the underlying principal with all CDMA techniques. Because the sensor data value being measured is relatively static, over many samples a very high precision measurement will converge close to the true sensor value for relay to the downstream analytics software.

The data obtained from thousands of colony member tags are harvested by the colony coordinators and further the data from thousands of Colonies where the System organizes groups of these Colonies each reporting chemical, biological and temperature information into groups of Colonies where the System decodes the temperature chemical and biological information and processes the information from each colony member in a time correlated database to form an electronic digital domain representation of chemical, biological and temperature information for further analysis.

The System also formats the time series database based on the interpreted chemical and biological information for relay back to a decision point thus creating actionable intelligence of decay at the carton and item level.

Thereby using the above, Apparatus, Method and System collecting data from inside an electrical dielectric medium of interest for the temperature, concentration of a chemical or biological analyte located physically inside each carton or item and relaying this data to external information processes the object of the invention is thus fulfilled.

The terminology used herein is for the purpose of describing particular embodiments only and is not intended to be limiting. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.” Moreover, unless specifically stated, any use of the terms first, second, etc., does not denote any order or importance, but rather the terms first, second, etc., are used to distinguish one element from another.

Unless otherwise defined, all terms (including technical and scientific terms) used herein have the same meaning as commonly understood by one of ordinary skill in the art to which embodiments of the invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

While various disclosed embodiments have been described above, it should be understood that they have been presented by way of example only, and not limitation. Numerous changes, omissions and/or additions to the subject matter disclosed herein can be made in accordance with the embodiments disclosed herein without departing from the spirit or scope of the embodiments. Also, equivalents may be substituted for elements thereof without departing from the spirit and scope of the embodiments. In addition, while a particular feature may have been disclosed with respect to only one of several implementations, such feature may be combined with one or more other features of the other implementations as may be desired and advantageous for any given or particular application. Furthermore, many modifications may be made to adapt a particular situation or material to the teachings of the embodiments without departing from the scope thereof.

Further, the purpose of the foregoing Abstract is to enable the U.S. Patent and Trademark Office and the public generally and especially the scientists, engineers and practitioners in the relevant art(s) who are not familiar with patent or legal terms or phraseology, to determine quickly from a cursory inspection the nature and essence of this technical disclosure. The Abstract is not intended to be limiting as to the scope of the present disclosure in any way.

Therefore, the breadth and scope of the subject matter provided herein should not be limited by any of the above explicitly described embodiments. Rather, the scope of the embodiments should be defined in accordance with the following claims and their equivalents.

1. A system comprising:
at least one resonator located within a first electrical dielectric medium where a product is located, the product emits at least one chemical as the product ages;
and transmitter located at a dielectric boundary of said first electrical dielectric medium to provide communications to an external analyzer of data emitted by the at least one resonator and to provide electrical energy to the resonator by way of radio waves.

2. The system according to claim 1, wherein the transmitter is located in a second electrical dielectric medium.

3. The system according to claim 1, wherein the at least one resonator comprises at least one temperature sensor.

4. The system according to claim 1, further comprising a housing providing a known dielectric medium around at least a part of the resonator, said housing further comprising an alignment component to counteract mechanical vibration experienced by the resonator.

5. The system according to claim 1, wherein said first electrical dielectric medium of interest has an electromagnetic wave number greater than or equal to three times said second dielectric medium of interest.

6. The system according to claim 1, wherein the resonator comprises a chemoresistive surface to sense a specific analytes concentration.

7. The system according to claim 1, wherein the transmitter comprises a first electrically small antenna tuned to radio frequencies of the at least one resonator in a form of a feed suitable for exciting an electrically dielectric resonant structure of the first electrical dielectric medium.

8. The system according to claim 1, wherein the transmitter emits a RF frequency to provide power to the at least one resonator.

9. A method comprising:
at least one of controlling, clocking and synchronizing a plurality of sensing RFID tags forming a colony inside an electrical dielectric medium;
communicating data acquired from at least one sensing RFID tag to a transmitter located external a dielectric environment where the plurality of sensing RFID tags are located;
synchronizing the data with the transmitter

10. The method according to claim 8, wherein synchronizing further comprises switching on/off an electromagnetic RF field that powers the plurality of sensing RFID tags.
